(19) United States
(12) Patent Application Publication
(10) Pub. No.: US 2008/0311215 A1
(43) Pub. Date: Dec. 18, 2008

(54) All-natural composition and method for repelling and killing insects

(76) Inventor: Catherine A. Grace, Marcellus, NY (US)

Correspondence Address: MARJAMA MULDOON BLASIAK & SULLIVAN LLP, 250 SOUTH CLINTON STREET, SUITE 300, SYRACUSE, NY, 13202, US

(21) Appl. No.: 11/818,615

(22) Filed: Jun. 18, 2007

(51) Int. Cl. A01N 65/00; A01N 61/00; A01P 17/00; A61K 35/64; A61K 36/00; A61K 36/534; A61P 43/00

(52) U.S. Cl. 424/537; 424/725; 424/747

(57) ABSTRACT

An all-natural composition can be used to repel or kill insects. The composition is an aqueous solution or mixture of available non-toxic ingredients. These include a weak acid to lower the pH, and one or more aromatics. The ingredients include one or more herbal essential oils, such as rosemary oil, peppermint oil, or a blend; a natural aqueous sugar extract or extracts, such as honey and/or molasses; a fragrance; and a natural weak acid solution, such as apple cider vinegar and/or lemon juice. The composition can be applied to dogs, horses, or other pets. The composition is also effective on hair and skin to combat the effects of swimming pool chlorine.

Applicant claims priority of Provisional Appln. No. 60/813,455, filed Jun. 15, 2006.

### BACKGROUND OF THE INVENTION

This invention relates to pest control arrangements, and is especially directed to a deterrent liquid or spray that contains only natural ingredients that are safe for application to skin and hair. The invention is more particularly concerned with an insect deterrent or agent that is effective against most flying or crawling insects.

Flies, mosquitos, moths and other insects such as cockroaches and ants are a constant nuisance to both humans and pets (dogs, horses, etc.). Currently, the only effective deterrent treatments have required the application of creams and sprays that contain synthetic organic chemicals, such as DEET. DEET is considered a suspected carcinogen. These existing treatments can be hazardous if ingested and can create a reaction in some persons. Also, these products can be expensive, if used extensively. They are also difficult to apply to dogs and other mammals (and birds), and generally repellent agents are not used on these animals.

### OBJECTS AND SUMMARY OF THE INVENTION

Accordingly, it is a desired object to provide an all natural composition that is an effective deterrent agent for flying insects (such as flies, mosquitos, moths, wasps, etc.) but can be applied to the skin and hair of humans or pets without creating adverse health effects.

It is a related object to provide an all natural composition that can be used as an insecticide that is safe for humans and pets.

It is another object to provide a repellent composition that also has a beneficial effect on the skin and hair or fur.

Accordingly, the invention can be embodied as an all-natural composition, safe for contact with human or pet skin and hair, comprising an herbal essential oil; a natural aqueous sugar extract or extracts; a fragrance; a natural weak acid solution and the balance an amount of pure water. Other ingredients may also be present, as discussed below.

I have discovered an all-natural composition that can be safely applied to human skin and hair. When the composition is applied to skin and hair, it acts as a natural insect repellent and deters mosquitos, black flies, horse flies, and other pests from alighting. If sprayed directly onto the insect, or sprayed as a mist in the area where the insect is flying, the product will stop the insect, i.e., kill or paralyze the insect. The product has no adverse affects on birds or mammals, and does not appear to affect other vertebrates. I have also found that this composition may be used after swimming in a chlorinated pool, and will neutralize the effects of the chlorine on the hair. Also, the use of this product will add body and luster to the hair or fur. If applied regularly to the face and neck, the product will soften and beautify the user's skin and complexion.

The composition can be used to repel insects, especially ants and cockroaches, from living quarters for prolonged periods of time, safely and effectively. The composition can be sprayed (either as a mist or as a stream) into a zone where the insects are present or where they are likely to be present. The composition can be safely used as an insect repellent for people as well as animals.

Generally, the composition is an aqueous solution or mixture of available non-toxic ingredients, including a weak acid to lower the pH, and one or more aromatics. To make up a batch of the product of about 250 ml volume, an herbal essential oil, such as rosemary oil, peppermint oil, or a blend (2 to 5 grams), a natural aqueous sugar extract or extracts, such as honey and/or molasses (2 to 5 grams), a fragrance (2 to 5 grams), and a natural weak acid solution (125 ml) are blended with sufficient amount of pure water to make up 250 ml. The essential herbal oil may be rosemary oil, or another similar oil. The acid solution may be organic apple cider vinegar (i.e., an acetic acid solution), but lemon juice or other citric fruit product (i.e., a citric acid solution) could be added. Examples of sugar extracts are molasses and honey, but other natural sugar solutions exist. When apple cider vinegar is used, the added fragrance can be an apple fragrance. Additional ingredients can be added, or these ingredients modified, to reduce odor or to reduce the pungency of the vinegar or other acid solution, if that is desired. Citric acid does reduce some of the pungency of the vinegar. Aloe may be added as a skin soothant.

### DESCRIPTION OF THE PREFERRED EMBODIMENTS

One specific example of the product, in a volume of 8 fluid ounces, is as follows: 3½ oz. spring water; ½ cup organic apple cider vinegar (ACV); ½ teaspoon honey; ½ teaspoon molasses; 1 tsp apple fragrance; ½ teaspoon rosemary oil. This simple blend is effective as a repellent of flying and crawling insects, but also has an enhancing effect on the skin and hair, as discussed above. The pungent odor of the essential oils and vinegar can be masked or eliminated by adding a natural fragrance.

A second example, embodying a repellent formulation, is blended as follows: 3 tbs rosemary oil; 1 tbs lemon juice; 2 tbs+1 tsp aloe; 2 tsp honey; 2 tsp molasses; 1 tbs glycerine; 1 cup ACV; and sufficient pure water to make up 16 ounces of the product (nominally, ½ cup water).

A third example, similar to the second, constitutes eight ounces of product: 1 tbs rosemary oil; 1 tsp peppermint oil; 2 tsp lemon juice; 1 tsp glycerine; 1 tsp aloe; 1 tsp honey; 1 tsp molasses; ½ cup+1 tbs+1 tsp ACV; and sufficient water to make up eight ounces.

The examples two and three have been particularly effective when sprayed onto the fur of dogs in keeping away flies and other insects.

A fourth example is an effective spray for temporarily paralyzing insects when the product is sprayed directly onto the insect or when it is sprayed as a mist in the zone where the insect is flying, or may be sprayed as a stream directly on the insect:

Peppermint oil—2 tsp; Rosemary oil—3 tsp; Lemon juice—2 tsp; Honey—2 tsp; Molasses—2 tsp; Glycerine—1 drop; Hydrogenated Vegetable Oils—1 drop; Apple Cider Vinegar—½ cup; White Vinegar—¼ cup; and sufficient water to make up eight ounces (nominally, 1 tsp). In this example, the peppermint oil, rosemary oil, lemon juice are considered the active ingredients, and respectively make up substantially 3.6%, 5.4%, and 6.6% of the composition, by weight. This product can be sprayed in kitchen cabinets or elsewhere that food is stored or prepared. The ingredients are all natural, non-toxic, non-staining and eco-friendly.

One important use of this product (i.e., fourth example) is to repel ants and cockroaches in areas of the home. Wherever it is sprayed, i.e., kitchen, bathroom, basement, etc., the insects disappear and do not return for many months. One user in Brooklyn, N.Y., reports that after spraying her kitchen with this product, her kitchen has been free of roaches for seven months. Another user, in Marcellus, N.Y., reports that she had an infestation of ants in her kitchen, but after using this product (fourth example) her kitchen was free of ants for at least two months.

The effect of this product on ants makes the product particularly attractive to owners of log homes, which are often plagued by carpenter ants.

Of course, the blends and proportions can be changed, and other essential oils can be used in place of peppermint oil and rosemary oil. Other weak acid solutions and other natural sugar sources can be used, if desired, and additional ingredients may be included.

The foregoing formulations given as specific embodiments of my composition are meant to serves as a few typical examples only. The person skilled in this field can vary the formulation, and can vary the techniques of application, without departing from the scope and spirit of my invention, which is given in the appended claims.

1. A natural composition that can be safely applied to skin and hair of a human or other mammal, and which is effective as a deterrent against insect pests, the composition comprising an herbal essential oil; a natural aqueous sugar extract or extracts; a fragrance; a natural weak acid solution; and water.

2. The composition of claim 1 wherein said herbal essential oil comprises at least one of rosemary oil and peppermint oil.

3. The composition of claim 1 wherein said natural aqueous sugar solution or extract comprises at least one of honey and molasses.

4. The composition of claim 1 wherein said weak acid solution comprises at least one of an acetic acid aqueous solution and a citric acid aqueous solution.

5. The composition of claim 4 wherein said acetic acid aqueous solution comprises at least one of apple cider vinegar and white vinegar.

6. The composition of claim 4 wherein said citric acid aqueous solution comprises lemon juice.

7. (canceled)

8. The composition of claim 1 comprising a formulation of rosemary oil; lemon juice; aloe; honey; molasses; glycerine; apple cider vinegar; and water.

9. The composition of claim 1 comprising a formulation of rosemary oil; peppermint oil; lemon juice, glycerine; aloe; honey; molasses; apple cider vinegar; and water.

10. (canceled)

11. A process of deterring flying or crawling insects, the process including spraying a composition into a zone where the insects are or may be present, the composition comprising: an herbal essential oil; a natural aqueous sugar solution or extract; a natural weak acid solution; and water.

12. A process of treating hair and skin to combat the effects of swimming pool chlorine, the process comprising, after first exposing a person's hair and skin to swimming pool chlorine while swimming; the step of applying to the person's skin and hair, following swimming, a composition comprising an herbal essential oil; a natural aqueous sugar extract or extracts; a fragrance; a natural weak acid solution; and the balance an amount of pure water.

13. The process of claim 11 wherein the composition further comprises at least one of glycerine, hydrogenated vegetable oil, and aloe.

14. The process of claim 11 further comprising applying the composition to the skin and hair of a human or other mammal.

15. The process of claim 11 wherein the herbal essential oil comprises at least one of rosemary oil and peppermint oil, wherein the natural aqueous sugar solution or extract comprises at least one of honey and molasses, and wherein the natural weak acid is at least one of apple cider vinegar, white vinegar, and lemon juice.

16. The process of claim 15 wherein the composition further comprises at least one of glycerine, hydrogenated vegetable oil, and aloe.

17. The process of claim 15 further comprising applying the composition to the skin and hair of a human or other mammal.

18. The process of claim 11 wherein the herbal essential oil comprises a blend of the rosemary oil and the peppermint oil, wherein the aqueous sugar solution or extract comprises a blend of the honey and the molasses, and wherein the weak acid solution comprises a blend of the apple cider vinegar and lemon juice.

19. The process of claim 18 wherein the weak acid solution further comprises white vinegar.

20. The process of claim 18 wherein the composition further comprises at least one of glycerine, hydrogenated vegetable oil, and aloe.

21. The process of claim 18 further comprising applying the composition to the skin and hair of a human or other mammal.
